(19) United States
(12) Patent Application Publication
(10) Pub. No.: US 2025/0127615 A1
(43) Pub. Date: Apr. 24, 2025

(54) PROSTHETIC HEART VALVES AND PROSTHETIC HEART VALVE DELIVERY ASSEMBLIES

(71) Applicant: EDWARDS LIFESCIENCES CORPORATION, Irvine, CA (US)

(72) Inventors: Ikechukwu Uzoma Okafor, Irvine, CA (US); Emily Cheng Zhou, Aliso Viejo, CA (US)

(21) Appl. No.: 18/999,565

(22) Filed: Dec. 23, 2024

Related U.S. Application Data
(63) Continuation of application No. PCT/US2023/028328, filed on Jul. 21, 2023.
(60) Provisional application No. 63/369,170, filed on Jul. 22, 2022.

(51) Int. Cl. A61F 2/24

(52) U.S. Cl. CPC A61F 2/2418; A61F 2/2436; A61F 2220/0008; A61F 2220/0025

(57) ABSTRACT

In some examples, a prosthetic heart valve for implantation within a native heart valve includes a radially expandable frame and a valvular structure. The frame includes an annular main body having an inflow end and an outflow end. The main body is radially expandable between a radially compressed state and a radially expanded state. The valvular structure includes a plurality of leaflets disposed within and coupled to the frame. The frame includes a plurality of frame anchors coupled to the main body. Each frame anchor is configured to be positioned on or around a pair of free edges of two of the native leaflets of the native heart valve. In some examples, a prosthetic heart valve delivery assembly includes a prosthetic heart valve and a delivery apparatus for delivering the prosthetic heart valve to an implantation site.

### CROSS REFERENCE TO RELATED APPLICATION

This application is a continuation of PCT Patent Application No. PCT/US2023/028328 filed on Jul. 21, 2023, which claims the benefit of U.S. Provisional Patent Application No. 63/369,170, filed on Jul. 22, 2022, each of these applications being incorporated by reference herein in its entirety.

### FIELD

The present disclosure concerns examples of a prosthetic valve (for example, a prosthetic heart valve) and a delivery apparatus for implanting a prosthetic valve.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally invasive surgical approaches are used in various procedures to deliver prosthetic medical devices to locations inside the body that are not readily accessible by surgery or where access without surgery is desirable.

In one specific example, a prosthetic heart valve can be mounted in a crimped state on the distal end of a delivery apparatus and advanced through the patient's vasculature (for example, through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

Balloon-expandable prosthetic valves typically are preferred for replacing calcified native valves because the catheter balloon can apply sufficient expanding force to anchor the frame of the prosthetic valve to the surrounding calcified tissue. On the other hand, self-expanding prosthetic valves sometimes are preferred for replacing a defective, non-stenotic (non-calcified) native valve, such as an insufficient native valve, although they also can be used to replace stenotic valves.

One problem associated with implanting a self-expandable prosthetic valve in a non-stenotic native valve is that the prosthetic valve may not be able to exert sufficient force against the surrounding tissue to resist migration of the prosthetic valve. Typically, the frame of the self-expandable prosthetic valve must be relatively long so that an upper portion of the frame can assist in anchoring the prosthetic valve within the descending aorta. Unfortunately, the upper portion of the frame can block access to the coronary ostia if future intervention is required. Further, if it becomes necessary to remove the prosthetic valve from the patient, portions of the frame may become fixed to non-diseased areas of the aorta, which can complicate removal of the prosthetic valve. Alternatively, the frame can have additional anchoring devices, which can be difficult to position and deploy.

### SUMMARY

Described herein are prosthetic heart valves, delivery apparatus, and methods for implanting prosthetic heart valves. The disclosed prosthetic heart valves, delivery apparatus, and methods can, for example, provide various features for maintaining the position and/or orientation of the prosthetic heart valve relative to a native annulus of a heart. As such, the devices and methods disclosed herein can, among other things, overcome one or more of the deficiencies of typical prosthetic heart valves and their delivery apparatus.

A prosthetic heart valve can comprise a frame and a valvular structure coupled to the frame. In addition to these components, a prosthetic heart valve can further comprise one or more of the components disclosed herein.

In some examples, a prosthetic heart valve can comprise a radially expandable frame comprising an annular main body having an inflow end and an outflow end, and the main body can be radially expandable between a radially compressed state and a radially expanded state.

In some examples, the frame can comprise a plurality of frame anchors coupled to the main body.

In some examples, the prosthetic heart valve can be configured for implantation within a native heart valve comprising a plurality of native leaflets, each frame anchor can be configured to be positioned on or around a pair of free edges of two of the native leaflets.

In some examples, the frame anchors can be configured to engage the native leaflets to anchor the prosthetic heart valve in position relative to an annulus of the native heart valve against retrograde blood flow.

In some examples, the main body can comprise an inflow end portion that comprises the inflow end, and the inflow end portion can be flared radially outward.

In some examples, each frame anchor can be configured to transition from a delivery configuration to a deployed configuration to anchor the prosthetic heart valve within the native heart valve, and each frame anchor can be biased toward the deployed configuration.

In some examples, each frame anchor is substantially U-shaped when the frame anchor is in one or both of the deployed configuration and the delivery configuration.

In some examples, each frame anchor can comprise an intermediate portion and a pair of leg portions extending away from the intermediate portion, and the prosthetic heart valve can be configured such that, when the prosthetic heart valve is implanted within the native heart valve, each leg portion engages a native leaflet of the native heart valve proximate to a native commissure of the native heart valve.

In some examples, the pair of leg portions can comprise a first leg portion and a second leg portion, and one or both of the first leg portion and the second leg portion can comprise an aperture that is configured to engage a cord of a delivery apparatus.

In some examples, a prosthetic heart valve for implantation within a native heart valve comprising a plurality of leaflets comprises a radially expandable frame and a valvular structure comprising a plurality of leaflets disposed within and coupled to the frame. The frame comprises an annular main body having an inflow end and an outflow end. The main body is radially expandable between a radially compressed state and a radially expanded state. The valvular structure is configured to regulate a flow of blood in one direction through the frame from the inflow end to the outflow end. The frame comprises a plurality of frame anchors coupled to the main body. Each frame anchor is configured to be positioned on or around a pair of free edges of two of the native leaflets.

In some examples, a prosthetic heart valve for implantation within a native heart valve comprising a plurality of leaflets comprises a radially expandable frame and a valvular structure comprising a plurality of leaflets disposed within and coupled to the frame. The frame comprises an annular main body having an inflow end and an outflow end. The main body is radially expandable between a radially compressed state and a radially expanded state. The valvular structure is configured to regulate a flow of blood in one direction through the frame from the inflow end to the outflow end. The frame comprises a plurality of frame anchors coupled to the main body. Each frame anchor comprises a first leg portion, a second leg portion, and an intermediate portion. Each of the first leg portion and the second leg portion extends away from the intermediate portion. Each frame anchor is configured to bend relative to the main body to transition between a delivery configuration and a deployed configuration. For each frame anchor, when the frame anchor is in the deployed configuration and when the prosthetic heart valve is implanted within the native heart valve, the first leg portion extends alongside a first leaflet of the plurality of native leaflets, the second leg portion extends alongside a second leaflet of the plurality of native leaflets, and the intermediate portion is positioned adjacent to free edges of each of the first leaflet and the second leaflet.

In some examples, a prosthetic heart valve comprises one or more of the components recited in Examples 1-52 below.

A prosthetic heart valve delivery assembly can comprise a handle and one or more shafts coupled to the handle. In addition to these components, a prosthetic heart valve delivery assembly can further comprise one or more of the components disclosed herein.

In some examples, a prosthetic heart valve delivery assembly can comprise a prosthetic heart valve for implantation within a native heart valve comprising a plurality of native leaflets and a delivery apparatus for delivering the prosthetic heart valve to an implantation site.

In some examples, the prosthetic heart valve can comprise a radially expandable frame and a valvular structure comprising a plurality of leaflets disposed within and coupled to the frame.

In some examples, the frame can comprise an annular main body having an inflow end and an outflow end.

In some examples, the valvular structure can be configured to regulate a flow of blood in one direction through the frame from the inflow end to the outflow end.

In some examples, the frame can comprise a plurality of frame anchors coupled to the main body.

In some examples, each frame anchor can be configured to be positioned on or around a pair of free edges of two of the native leaflets.

In some examples, the delivery apparatus can comprise a delivery capsule configured to house the prosthetic heart valve in a radially compressed state and a plurality of cords.

In some examples, each cord of the plurality of cords can be configured to be releasably connected to a respective frame anchor of the prosthetic heart valve and to apply a restraining force to the respective frame anchor.

In some examples, for each cord and the respective frame anchor, the frame anchor can comprise an aperture, and the cord can extend through the aperture when the prosthetic heart valve is housed within the delivery capsule.

In some examples, each frame anchor can be configured to transition from a delivery configuration to a deployed configuration to anchor the prosthetic heart valve within the native heart valve.

In some examples, for each cord and the respective frame anchor, the cord can be configured to maintain the frame anchor in the delivery configuration while the cord applies the restraining force to the frame anchor, and the frame anchor can automatically transition from the delivery configuration to the deployed configuration when the cord no longer applies the restraining force to the frame anchor.

In some examples, a prosthetic heart valve delivery assembly for comprises a prosthetic heart valve for implantation within a native valve comprising a plurality of native leaflets and a delivery apparatus for delivering the prosthetic heart valve to an implantation site. The prosthetic heart valve comprises a radially expandable frame and a valvular structure. The frame comprises an annular main body having an inflow end and an outflow end. The main body is radially expandable between a radially compressed state and a radially expanded state. The valvular structure comprises a plurality of leaflets disposed within and coupled to the frame and configured to regulate a flow of blood in one direction through the frame from the inflow end to the outflow end. The frame comprises a plurality of frame anchors coupled to the main body. Each frame anchor is configured to be positioned on or around a pair of free edges of two of the native leaflets. The delivery apparatus comprises a delivery capsule configured to house the prosthetic heart valve in a radially compressed state and a plurality of cords. Each cord is configured to be releasably connected to a respective frame anchor of the prosthetic heart valve and to apply a restraining force to the respective frame anchor.

In some examples, a prosthetic heart valve delivery assembly comprises one or more of the components recited in Examples 53-63 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE DRAWINGS

FIG. 1 is a perspective view of a prosthetic valve that can be used to replace a native valve of the heart.

FIG. 2 is a perspective view of another prosthetic valve that can be used to replace a native valve of the heart, according to one example.

FIG. 3 is a perspective view of a frame of the prosthetic valve of FIG. 2.

FIG. 4 is a top view of the frame of the prosthetic valve of FIG. 2.

FIG. 5 is a cross-sectional view of the heart showing the prosthetic valve of FIG. 2 implanted within the aortic annulus.

FIG. 6 is a top view of the prosthetic valve of FIG. 2 implanted within the aortic annulus of the heart as viewed along the line 6-6 in FIG. 5.

FIG. 7A is a side view of the frame of the prosthetic valve of FIG. 2 in an expanded, delivery state.

FIG. 7B is an enlarged side view of a frame anchor of the frame of FIG. 7A in a delivery configuration.

FIG. 7C is an enlarged side view of the frame anchor of FIG. 7B in a deployed configuration.

FIG. 8A is a side view of a frame of a prosthetic valve in an expanded, delivery state, according to another example.

FIG. 8B is an enlarged side view of a frame anchor of the frame of FIG. 8A in a delivery configuration.

FIG. 8C is an enlarged side view of the frame anchor of FIG. 8B in a deployed configuration.

FIG. 9A is a side view of the frame of FIG. 8A shown in a compressed state within a delivery capsule of a delivery apparatus, according to one example.

FIG. 9B is a side view of the delivery apparatus of FIG. 9A with the delivery capsule partially retracted.

FIG. 10A is a side view of the frame of FIG. 8A shown in a compressed state within a delivery capsule of a delivery apparatus, according to another example.

FIG. 10B is a side view of the delivery apparatus of FIG. 10A with the delivery capsule partially retracted.

FIG. 11 is a side view of a frame anchor in a deployed configuration and covered by a protective covering.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

As used herein, the term “substantially” means the listed value and/or property and any value and/or property that is at least 75% of the listed value and/or property. Equivalently, the term “substantially” means the listed value and/or property and any value and/or property that differs from the listed value and/or property by at most 25%. For example, “at least substantially parallel” refers to directions that are fully parallel, and to directions that diverge by up to 22.5 degrees.

As used herein, “e.g.” means “for example,” and “i.e.” means “that is.”

### Examples of the Disclosed Technology

Described herein are examples of an implantable, expandable medical device, such as a prosthetic heart valve. The prosthetic heart valve is configured to be implanted in a native annulus of a patient's heart. To maintain the position and/or orientation of the prosthetic heart valve relative to the native annulus, the prosthetic heart valve comprises a flared inflow end configured to engage the native annulus and a plurality of frame anchors configured to engage the native leaflets of the native valve adjacent the commissures of the native leaflets. The prosthetic heart valve is particularly suited for implantation within a native aortic or pulmonic valve. However, the prosthetic heart can also be adapted to be implanted in other native valves of the heart (the mitral and tricuspid valves).

Prosthetic valves disclosed herein can be radially compressible and expandable between a radially compressed state and a radially expanded state. Thus, the prosthetic valves can be crimped on or retained by an implant delivery apparatus in the radially compressed state during delivery, and then expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

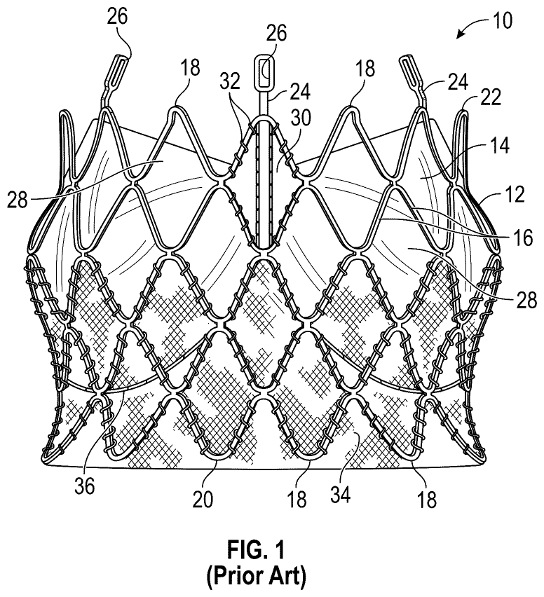

FIG. 1 shows an exemplary prosthetic valve 10, according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated by reference herein. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated by reference herein.

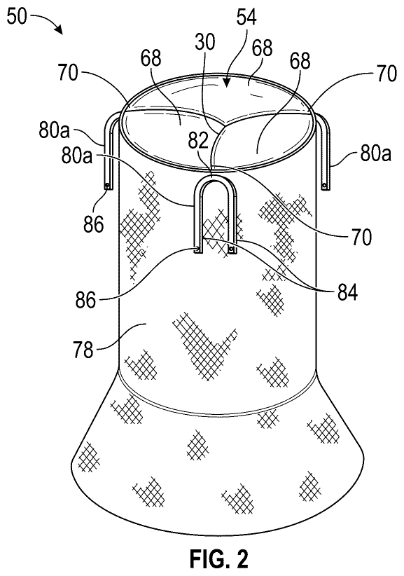

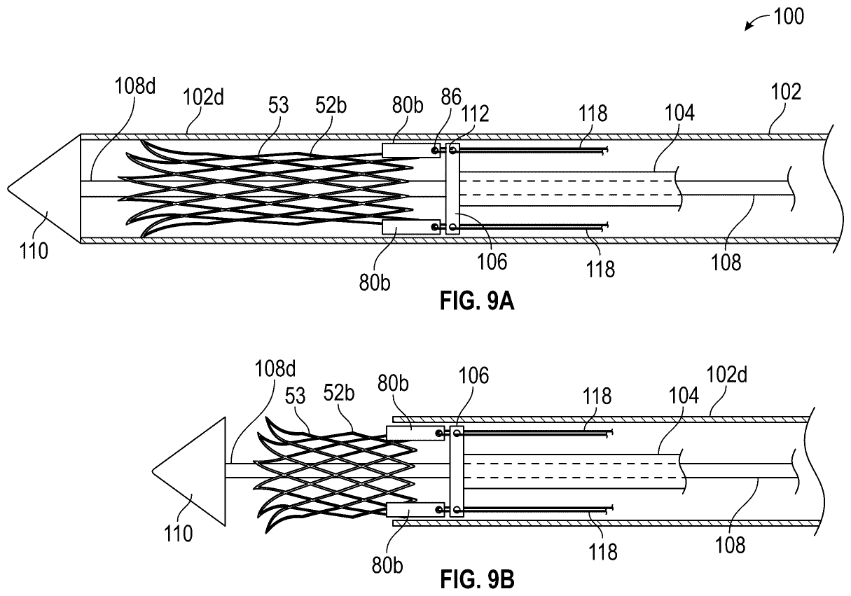

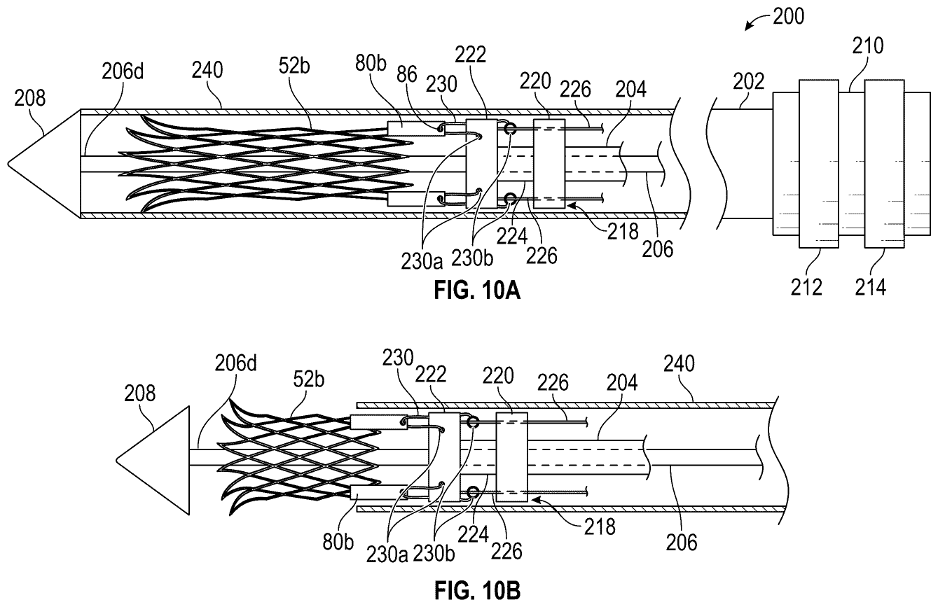

As discussed above, FIG. 1 shows a prosthetic heart valve 10, which is an example of a prosthetic heart valve as known in the art, while FIGS. 2-4 illustrate aspects of a prosthetic heart valve 50 according to the present disclosure. In some examples, the prosthetic heart valve (for example, the prosthetic heart valve 10 and/or the prosthetic heart valve 50) is a self-expanding valve that is delivered in a radially compressed state to a deployment site via a delivery apparatus. When advanced from a delivery capsule at the distal end of the delivery apparatus (as discussed with reference to FIGS. 9A-10B below), the prosthetic valve can radially self-expand to its functional size.

As shown in FIG. 1, the prosthetic heart valve 10 comprises a stent or frame 12 and a valvular structure 14 (for example, leaflets or a flap valve) supported by the frame. The frame 12 can have a plurality of interconnected and circumferentially extending struts 16 arranged in a lattice-like pattern and forming a plurality of apices 18 at the inflow end 20 and the outflow end 22, respectively, of the frame 12. The valvular structure 14 is configured to regulate a flow of blood through the prosthetic heart valve 10 from the inflow end 20 to the outflow end 22.

The frame 12 can include a plurality of angularly-spaced posts 24 extending from respective apices 18 at the outflow end of the frame 12. The frame 12 in the illustrated example includes three such posts 24, although a greater or fewer number of posts can be used. In one implementation, the frame 12 can have posts extending from all the apices 18 at the outflow end of the frame. Each post 24 can have an eyelet or aperture 26, which can be used to form a releasable connection with a delivery apparatus, such as via the use of one or more cords or tethers 118 (see FIGS. 9A-9B), as further described below.

In other examples, the apertures 26 can be formed at the inlet (or inflow) end 20 of the frame 12 where other delivery apparatus configurations or other delivery techniques require apertures at the inlet end of the frame, such as a transapical delivery approach.

The frame 12 can be made of any of various suitable plastically-expandable materials (for example, stainless steel, etc.) or self-expanding materials (for example, Nitinol) as known in the art. When constructed of a plastically-expandable material, the frame 12 (and thus the prosthetic heart valve 10) can be crimped to a radially compressed state on a delivery catheter and then expanded inside a patient by an inflatable balloon or equivalent expansion mechanism. When constructed of a self-expandable material, the frame 12 (and thus the prosthetic heart valve 10) can be crimped to a radially compressed state and restrained in the compressed state by insertion into a sheath or equivalent mechanism of a delivery catheter. Once inside the body, the prosthetic heart valve can be advanced from the delivery sheath, which allows the prosthetic heart valve to expand to its functional size.

Suitable plastically-expandable materials that can be used to form the frames disclosed herein (for example, the frame 12) include, metal alloys, polymers, or combinations thereof. Example metal alloys can comprise one or more of the following: nickel, cobalt, chromium, molybdenum, titanium, or other biocompatible metal. In some examples, the frame 12 can comprise stainless steel. In some examples, the frame 12 can comprise cobalt-chromium. In some examples, the frame 12 can comprise nickel-cobalt-chromium. In some examples, the frame 12 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

In particular examples, the prosthetic heart valve 10 is a self-expandable heart valve wherein the frame 12 is a made of a super-elastic, self-expanding material (for example, a nickel titanium alloy such as Nitinol) as is known in the art. When used with a delivery apparatus 100 (FIGS. 9-10), the prosthetic valve 10 can self-expand from a radially compressed state to a radially expanded state when advanced from a delivery capsule (for example, a delivery sheath) of the delivery apparatus. In particular, FIG. 1 illustrates the prosthetic valve 10 in the expanded state.

The valvular structure 14 can comprise a plurality of leaflets 28. The valvular structure typically comprises three leaflets 28 arranged in a tricuspid arrangement, although a greater or fewer number of leaflets 28 can be used. The leaflets 28 can be made of any various suitable materials, including natural tissue (for example, bovine pericardium or pericardium from other sources) or synthetic materials (for example, polyurethane). Adjacent side portions at the outflow edges (the upper edges in the drawings) of adjacent leaflets can be secured to each other to form commissures 30 of the valvular structure, which can be secured to the frame with sutures 32.

The prosthetic valve 10 can further include an inner skirt 34 mounted on the inside of the frame 12. The skirt 34 helps establish a seal with the surrounding tissue after implantation. The skirt 34 can also be used to mount portions of the leaflets 28 to the frame 12. For example, in the illustrated example, the inflow edges of the leaflets (the lower edges in the drawings) can be sutured to the skirt 34 along suture line 36. The skirt 34 can be connected directly to the frame 12, such as with sutures. Although not shown, the prosthetic valve 10 can include an outer skirt mounted on the outside of the frame in lieu of or in addition to the inner skirt 34 to further seal the prosthetic valve against surrounding tissue.

The inner and/or outer skirt can be wholly or partly formed of any suitable biological material, synthetic material (for example, any of various polymers), or combinations thereof. In some examples, the inner and/or outer skirt can comprise a fabric having interlaced yarns or fibers, such as in the form of a woven, braided, or knitted fabric. In some examples, the fabric can have a plush nap or pile. Exemplary fabrics having a plus nap or pile include velour, velvet, velveteen, corduroy, terrycloth, fleece, etc. In some examples, the inner and/or outer skirt can comprise a fabric without interlaced yarns or fibers or randomly interlaced yarns or fibers, such as felt or an electrospun fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide etc. In some examples, the inner and/or outer skirt can comprise a non-textile or non-fabric material, such as a film made from any of a variety of polymeric materials, such as PTFE, PET, polypropylene, polyamide, polyetheretherketone (PEEK), polyurethane (such as thermoplastic polyurethane (TPU)), etc. In some examples, the inner and/or outer skirt can comprise a sponge material or foam, such as polyurethane foam. In some examples, the inner and/or outer skirt can comprise natural tissue, such as pericardium (for example, bovine pericardium, porcine pericardium, equine pericardium, or pericardium from other sources).

Exemplary configurations of the prosthetic heart valve are further disclosed in WIPO Publication No. WO2021/195090 and in U.S. Patent Application Publication Nos. 2014/0343670, 2012/0123529, 2010/0036484, and 2010/0049313, the disclosures of which are incorporated by reference herein.

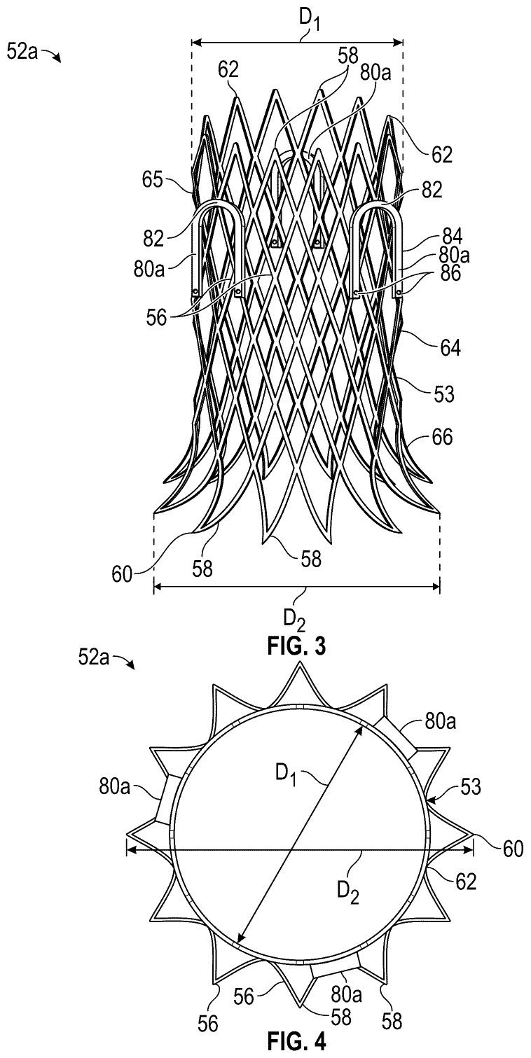

FIG. 2 illustrates a prosthetic heart valve 50 according to the present disclosure, while FIGS. 3-4 illustrates a stent or frame 52a of the prosthetic heart valve 50. In particular, FIGS. 2-4 illustrate the prosthetic heart valve 50 and/or the frame 52a in a radially expanded state. As used herein, the terms “expanded state” and/or “compressed state” may be used to refer to either or both of the prosthetic heart valve 50 and/or the frame 52a.

Unless otherwise specified, the prosthetic heart valve 50 and/or the frame 52a may share any suitable components, properties, features, etc. with the prosthetic heart valve 10 of FIG. 1. For example, and as illustrated in FIGS. 2-4, the frame 52a can include a base frame structure or main body 53 that includes a plurality of circumferentially extending struts 56, apices 58, an inflow end 60, and an outflow end 62, each of which may share any properties with the struts 16, the apices 18, the inflow end 20, and/or the outflow end 22, respectively, of the frame 12. Similarly, and as shown in FIGS. 2-4, the prosthetic heart valve 50 may include a valvular structure 54 comprising a plurality of leaflets 68 forming a plurality of commissures 70, which may share any properties with the valvular structure 14, the leaflets 28, and the commissures 30, respectively, of the prosthetic heart valve 10. The leaflets 68 may be assembled to the frame 52a in the same manner as the leaflets 28 of the prosthetic valve 10. For example, the cusp edges of the leaflets 68 can be sutured to an inner skirt 34 along a suture line (not shown in FIG. 2). Also, the commissures of the leaflets 68 can connected to the frame 52a, such as by suturing commissure tabs of the leaflets to struts of the frame in the manner shown in FIG. 1. However, it should be understood that the leaflets 68 can be assembled to the frame using any known techniques or mechanisms known in the art.

Additionally, as shown in FIG. 2, the prosthetic heart valve 50 may include an outer skirt 78 mounted on the outside of the main body 53 of the frame, which may share any applicable features and/or properties with the inner skirt 34 of the prosthetic heart valve 10. In particular, the outer skirt 78 may be secured to an exterior of the main body 53, such as with sutures. When present, the outer skirt 78 may be configured to seal the prosthetic heart valve 50 against surrounding tissue. As shown in FIG. 2, the outer skirt 78 may cover all, or at least substantially all, of the exterior surface of the main body 53 (which is thus hidden in FIG. 2). In other examples, the outer skirt 78 can cover only a portion of the outer surface of the main body 53. For example, the outer skirt 78 can extend from the inflow end 60 of the frame to a location axially between the inflow end 60 and the outflow end 62. Desirably, the outer skirt 78 extends over and covers at least a flared inflow end portion of the frame for sealing against tissue of a native annulus.

As shown in FIG. 3, the frame 52a has a main body 53 that comprises an inflow end portion or region 66, an outflow end portion or region 65, and a waist portion or region 64 extending between the inflow end portion and outflow end portion. The outflow end portion 65 is generally cylindrical, while the inflow end portion 66 is outwardly flared, at least when the frame 52a is in the expanded state. In particular, and as shown in FIG. 3, when the frame 52a is in the expanded state, the inflow end portion 66 increases in diameter from the waist portion 64 to the inflow end 60. Specifically, the outflow end 62 may have a diameter D₁, and the inflow end 60 may have a diameter D₂ that is greater than D₁.

As more specific examples, D₂ may be at least at least 110% of D₁, at least 125% of D₁, at least 150% of D₁, at least 175% of D₁, at least 200% of D₁, or at most 225% of D₁, at most 180% of D₁, at most 160% of D₁, at most 140% of D₁, and/or at most 120% of D₁.

In particular examples, for a prosthetic valve intended for use in a 22-mm to 24-mm annulus, the diameter D₂ is about 28 millimeters (mm) to about 32 mm, with 30 mm being a specific example; and the diameter D₁ is about 24 mm to about 28 mm, with 26 mm being a specific example. In particular examples, the prosthetic valve has a length, as measured between the inflow end 60 and the outflow end 62, that is about 20 mm to about 24 mm, with 22 mm being a specific example.

In some examples, such as in the example of FIG. 3, the diameter of the main body 53 is constant or substantially constant (for example, to within 10%) along the outflow end portion 65 and/or the waist region 64. Additionally, or alternatively, the diameter of the main body 53 may gradually increase (for example, monotonically increase) from the diameter D₁ at the outflow end 62 to the diameter D₂ at the inflow end 60.

In alternative examples, the main body 53 of the frame can have various other shapes or configurations. For example, the outflow end portion 65 can be outwardly flared such that the diameter of the outflow end portion 65 increases in a direction extending from the waist region 64 to the outflow end 62. In still alternative examples, the main body 53 can have the same shape as the frame 12 of FIG. 1. In still other examples, the main body 53 can be cylindrical and can have a constant or substantially constant diameter from the inflow end 60 to the outflow end 62.

The increased diameter of the inflow end 60 may assist in anchoring the prosthetic heart valve 50 in the annulus of a native valve (for example, a native aortic valve) once implanted in the heart. In particular, D₂ desirably is greater than the diameter of the native annulus in which the prosthetic heart valve 50 is to be implanted. In this manner, the overall shape of the frame 52a assists in retaining the prosthetic heart valve 50 at the implantation site.

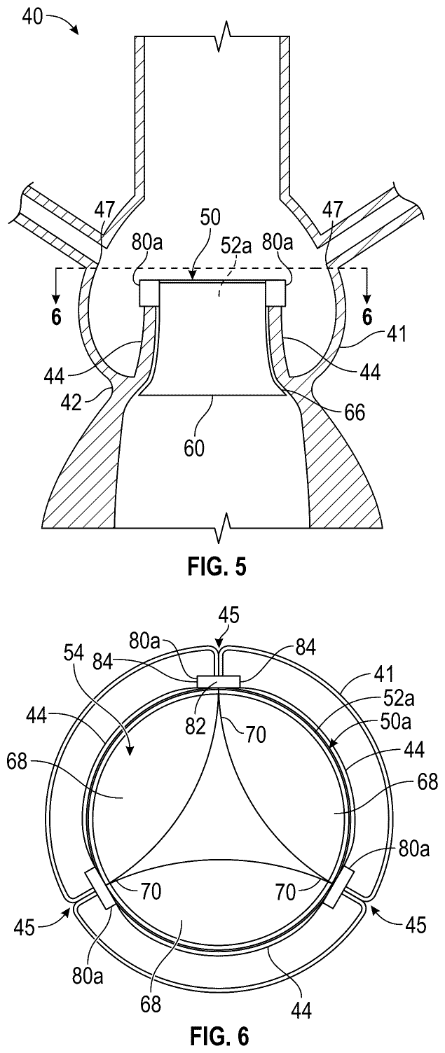

More specifically, FIGS. 5-6 illustrate the prosthetic heart valve 50 implanted within an aortic annulus 42 of an aorta 41 of a heart 40 such that the flared inflow end 60 extends below the aortic annulus 42. The prosthetic heart valve 50 is retained within the native valve at least in part by the radial outward force of the prosthetic heart valve 50 against the surrounding tissue of the aortic annulus 42 as well as the geometry of the frame 52a. Specifically, the flared inflow end region 66 is shaped to conform to the aortic annulus 42 and can extend to a sub-valvular location to better resist against axial dislodgement of the prosthetic heart valve 50 primarily in the upstream direction (toward the aorta). In certain examples, the inflow end 60 of the frame can be located in the left ventricle when the prosthetic valve 50 is implanted. In some examples, the radial outward force of the inflow end region 66 against the native annulus can also assist in retaining the prosthetic valve against axial dislodgement in the downstream direction (toward the left ventricle). Moreover, as shown in FIG. 5, the prosthetic heart valve 50 may form a substantially fluid-tight seal with the aortic annulus 42 via contact between the aortic annulus 42 and the flared inflow end region 66 of the frame 52a (and/or the outer skirt 78 within this region) to prevent or minimize paravalvular leakage.

The frame 52a of the prosthetic heart valve 50 additionally may include dedicated features and/or structures for anchoring the prosthetic heart valve 50 relative to the native aortic annulus 42. In particular, and as illustrated in FIGS. 2-4, the frame 52a of the prosthetic heart valve 50 comprises a plurality of frame anchors 80a coupled to the main body 53 at locations axially spaced from the inflow end of the frame. The frame anchors 80a are configured to engage the native leaflets 44 of the aortic valve at or adjacent the native commissures 45 to assist in retaining the prosthetic heart valve 50 in position relative to the aortic annulus 42 against the retrograde blood flow.

The number of frame anchors 80a desirably corresponds to the number of native commissures 45 of the native valve in which the prosthetic valve is to be implanted. For most patients, the native aortic valve, tricuspid valve and pulmonary valve have three native commissures. Thus, for implantation at a native aortic valve, tricuspid valve or pulmonary valve, the frame can have three frame anchors 80a corresponding to the three native commissures. For implantation at a bi-cuspid native aortic valve or a native mitral valve, the frame can have two frame anchors 80a.

In other examples, the frame 52a can have fewer frame anchors 80a than the number of native commissures of the native valve in which the prosthetic valve is to be implanted, such as one or two frame anchors 80a for a native valve having three commissures or one frame anchor 80a for a native valve having two commissures.

In still other examples, the frame 52 can have more frame anchors 80a than the number of native commissures of the native valve in which the prosthetic valve is to be implanted. Such a configuration may facilitate positioning a respective frame anchor 80a at each native commissure in a native valve in which the native commissures are not evenly spaced from each other in a circumferential direction. For example, the frame can have a plurality of primary frame anchors corresponding to the number of native commissures and one or more secondary frame anchors, each of which is positioned circumferentially between two primary frame anchors. If it is determined that one or more primary anchors do not align with a native commissure, the frame can be positioned such that one or more of the secondary anchors are aligned with those native commissures.

In the illustrated example, as shown in FIGS. 2 and 6, each frame anchor 80a is located on the main body of the frame at a position circumferentially aligned with a commissure 70 of the prosthetic valve. As such, when the prosthetic valve 50 is implanted within a native aortic valve, each frame anchor 80a is located at a native commissure 45 and each commissure 70 of the prosthetic valve is rotationally aligned with one of the native commissures 45. In this position, each commissure 70 of the prosthetic valve 50 is rotationally offset from the coronary ostia 47. In this manner, the frame anchors 80a serve as positioning/alignment members for rotationally aligning the prosthetic valve relative to the coronary ostia.

Positioning the commissures at locations rotationally offset from the coronary ostia 47 has several potential benefits. For example, a commissure of a prosthetic valve positioned in front of a coronary ostia can block or interfere with catherization of the coronary artery in a subsequent procedure. However, positioning the commissures 70 of the prosthetic valve at locations rotationally offset from the coronary ostia 47 can facilitate access the coronary arteries, such as for subsequent coronary angiographic or angioplasty procedures. Moreover, if a replacement prosthetic valve is implanted in a previously implanted prosthetic 50 (in a “valve-in-valve” procedure), it may be desirable to modify the leaflets 68 of the previously implanted valve, such as by cutting the leaflets 68 or forming openings in the leaflets 68 at locations between commissures, to increase access to the coronary arteries. A physician can more easily access the leaflets 68 with a medical device for modifying the leaflets when the previously implanted prosthetic valve is in this orientation.

In other examples, the frame anchors 80a can be positioned on the main body of the frame at locations circumferentially offset from the commissures 70.

The position of the native leaflets 44 relative to the frame 52a and/or the frame anchors 80a once the prosthetic valve 50 is implanted may vary depending on the patient's anatomy and/or the condition of the native leaflets 44. For simplicity, FIG. 6 illustrates each native leaflet 44 as contacting the outer surface of the frame 52a and/or the outer skirt 78, which can be the case for stenosed (calcified) native leaflets 44.

In other examples, however, each native leaflet 44 may extend relative to the frame anchors 80a and/or relative to the frame 52a in any of a variety of manners depending on the anatomy of the patient and the condition of the native leaflets. For example, each native leaflet 44 may extend between adjacent frame anchors 80a such that each native leaflet 44 is spaced apart in a radial direction from the outer surface of the frame anchors 80a and from the exterior surface of the frame 52a, at least in the region proximate to the outflow end 62, which can be the case for non-stenosed (non-calcified) native leaflets 44.

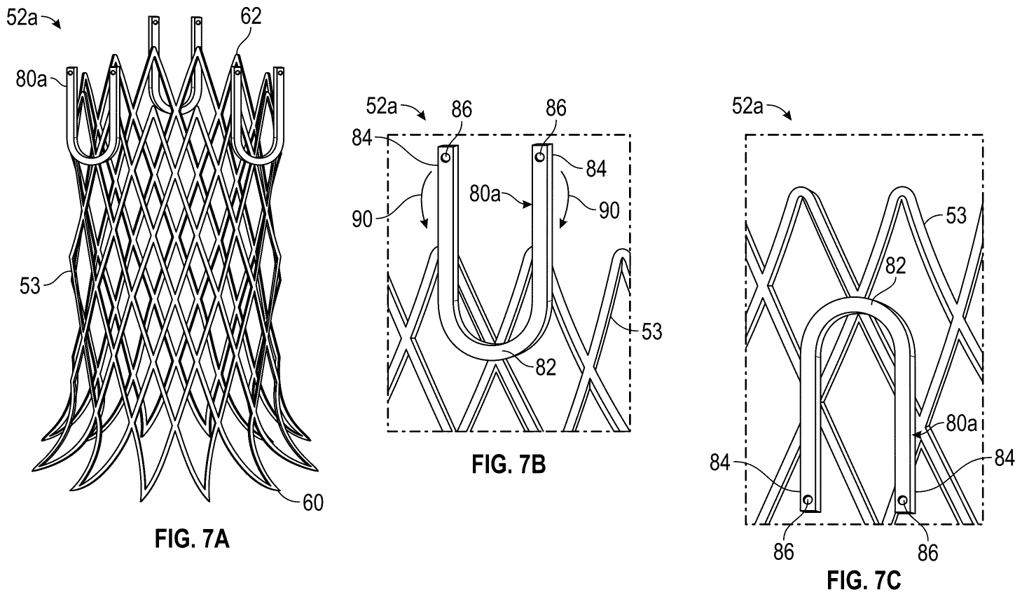

Each frame anchor 80a may be attached to the main body 53 of the frame 52a at any of a variety of locations, such as any locations along the waist portion 64 or the outflow end portion 65. In the example of FIGS. 3 and 7A-7C, each frame anchor 80a is attached to the main body 53 at an intersection of four struts 56 at a location that is spaced from each of the inflow end 60 and the outflow end 62. Specifically, in the example of FIGS. 3 and 7A-7C, each frame anchor 80a is attached to the main body 53 at a location that is spaced from the outflow end 62 by one row of struts 56. In other examples, each frame anchor 80a may be attached to the main body 53 at an intersection of struts 56 at the outflow end 62 (for example, at an outflow apex 58), or may be attached to a single strut 56.

In some examples, and as shown in FIGS. 3 and 7A, the frame anchors 80a may be evenly distributed around a circumference of the main body 53. In particular, in the example of FIGS. 3 and 7A, the main body 53 includes twelve outflow apices 58, and each frame anchor 80a is attached to the main body 53 between a corresponding pair of outflow apices 58 such that each pair of adjacent frame anchors 80a are separated by four outflow apices 58. This is not required, however, and it additionally is within the scope of the present disclosure that the frame anchors 80a may be unevenly distributed around a circumference of the main body 53.

Each frame anchor 80a may be coupled to the main body 53 in any suitable manner. In some examples, each frame anchor 80a is formed of the same material as the main body 53, and the frame anchor 80a may be integrally formed with the main body 53. For example, the main body 53 and the frame anchors 80a can be formed (for example, laser cut) from the same piece of material (for example, a piece of Nitinol). In other examples, each frame anchor 80a and the main body 53 can be separately formed and the frame anchors can be subsequently joined to the main body 53. Various techniques and mechanisms can be used to connect the frame anchors 80a to the main body 53, such as welding the frame anchors to the main body, adhesively bonding the frame anchor to the main body, or using mechanical connectors, such as sutures, screws, rivets, pins, or various other connection means.

In the example of FIG. 2, each frame anchor 80a is coupled to the main body 53 such that the outer skirt 78 extends at least partially between each frame anchor 80a and the main body 53. In particular, the outer skirt 78 may include holes, cutouts, openings, apertures, etc. at the locations at which each frame anchor 80a is attached to the main body 53.

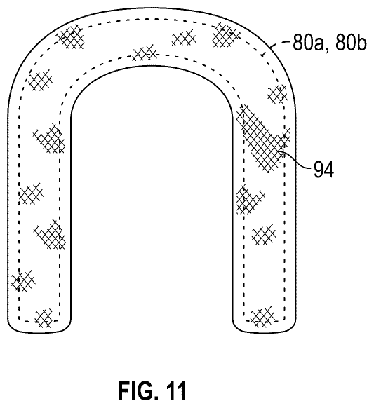

In some examples, and as shown in FIG. 11, each frame anchor 80a may be at least partially covered, or substantially covered by (for example, wrapped in) a protective covering 94 that protects the native leaflets from directly contacting the frame anchor 80a. In such examples, the protective covering 94 can minimize or prevent trauma to the patient's native tissues by avoiding direct contact between a metal anchor and the tissue by avoiding direct contact between a metal anchor and the tissue. When present, the protective covering 94 may include and/or be any of a variety of materials, examples of which include any of various synthetic materials or natural tissue. For example, the covering can be in the form of a fabric (such as PET fabric or fabrics formed from other types of synthetic fibers), or non-fabric materials, such as in the form of a layer of a polymeric material (such as a layer of thermoplastic polyurethane (TPU)) or a layer of foam or sponge (such as polyurethane foam or a hydrogel foam). Additionally or alternatively, the protective covering 94 may be formed from the same material as the inner and/or outer skirt of the prosthetic valve 10. This is not required of all examples, however, and it also is within the scope of the present disclosure that each frame anchor 80a is uncovered.

In the example of FIGS. 2-4, each frame anchor 80a comprises an intermediate portion 82 and a pair of leg portions 84 extending away from the intermediate portion 82. Thus, each frame anchor 80a in the illustrated example is U-shaped, or substantially U-shaped, although the anchors can have various other shapes in other examples, such a V-shape. In the illustrated example, the intermediate portion 82 can be fixed relative to the frame and therefore can be referred to as a fixed portion of the frame anchor 80a. The leg portions 84 can move (for example, pivot) between a delivery configuration (FIGS. 7A and 7B) and a deployed configuration (FIGS. 3 and 7C).

As shown in FIGS. 7A and 7B, in the delivery configuration, each leg portion 84 extends away from the intermediate portion 82 in a downstream direction toward the outflow end 62 of the frame. In some examples, when each frame anchor 80a is in the delivery configuration, each leg portion 84 may extend from the intermediate portion 82 toward the outflow end 62 and continue beyond the outflow end 62 of the frame. As shown in FIGS. 3 and 7C, in the deployed configuration, each leg portion 84 extends away from the intermediate portion 82 toward the inflow end 60 of the frame.

The detail views of FIGS. 7B-7C illustrate an example of a manner in which each frame anchor 80a may transition from the delivery configuration (FIG. 7B) to the deployed configuration (FIG. 7C). Specifically, in the example of FIGS. 7B-7C, each leg portion 84 pivots relative to the intermediate portion 82 as the frame anchor 80a transitions from the delivery configuration to the deployed configuration, as indicated by arrows 90. More specifically, each leg portion can be configured to pivot about an axis that is perpendicular to a plane that extends tangent to the main body 53 wherein the frame anchor 80a is attached to the main body 53. In such examples, each leg portion 84 may be integrally formed with the intermediate portion 82 such that the leg portions 84 can bend or deform when transitioning from the delivery configuration to the deployed configuration.

In certain examples, the frame anchors 80a can be formed from a shape-memory material, such as Nitinol. When formed from a shape-memory material, the anchors 80a can be shape set in the deployed configuration. Thus, when the prosthetic valve 50 is loaded onto a delivery apparatus for delivery into a patient's body, the frame anchors 80a can be deformed to the delivery configuration and retained in the delivery configuration by retaining elements (for example, tethers) of the delivery apparatus. When released from the retaining elements at or near the implantation site, the anchor members can automatically revert to the deployed configuration under their own resiliency.

As illustrated in FIGS. 2-3 and 7A-7C, each leg portion 84 may include an eyelet, or aperture, 86 that is configured to engage a tether of a delivery apparatus for selectively retaining the leg portion 84 in the delivery configuration against the inherent spring force that biases the leg portion to the deployed configuration. When the prosthetic valve is loaded onto a delivery apparatus, a tether can be routed through the aperture 86 of each leg portion and pulled in a proximal direction to bend the leg portions 84 to the delivery configuration. During delivery through a patient's vasculature, the tethers can be held taut to the retain the leg portions in the delivery configuration. While deploying the main body 53 of the frame (or alternatively, before or after deploying the main body 53), tension on the tethers can be released to allow the leg portions 84 to revert to the deployed configuration under their own resiliency, after which the tethers can be removed from the leg portions 84. Examples of delivery apparatuses and methods for implanting the prosthetic valve are further described in detail below.

In other examples, the frame anchors 80a can be formed from a plastically-deformable material, such as stainless steel, cobalt-chromium alloy, in which case the frame anchors 80a can be plastically deformed from the delivery configuration to the deployed configuration at or near the implantation site, such as by actuation of components of the delivery apparatus.

In the deployed configuration, each frame anchor 80a is configured to be placed around the free edges of a pair of native leaflets at or in close proximity to a native commissure. As discussed above, FIGS. 5-6 illustrate an example in which the prosthetic heart valve 50 is implanted within the native aortic annulus 42 of the heart 40. In the example of FIGS. 5-6, each frame anchor 80a is in the deployed configuration, and is placed around the free edges of a pair of native leaflets 44 at a native commissure 45. Thus, when the prosthetic heart valve 50 is implanted in the aortic annulus 42, for each frame anchor 80a, the intermediate portion 82 is positioned adjacent or against the free edges of a pair of leaflets 44, one of the leg portions 84 extends alongside one of the leaflets 44, and the other leg portion 84 extends alongside the other leaflet of the same native commissure 45. In this manner, the frame anchors 80a can assist in retaining the prosthetic valve within the native aortic valve by resisting migration of the prosthetic valve in an upstream direction (toward the left ventricle) against retrograde blood flow.

In some examples, the leg portions 84 can be configured to clamp onto a pair of native leaflets or pinch a pair of leaflets between the leg portions. In other words, the lateral spacing between leg portions 84 can be sized such that when a frame anchor is placed around a pair of leaflets, the leg portions 84 exert a clamping force against the adjacent surfaces of the leaflets. In this manner, the leg portions 84 engage and frictionally engage the leaflets between the leg portions via the clamping force exerted by the leg portions against the leaflets. If the frame anchor is formed a shape-memory material, the leg portions 84 can be configured to clamp onto a pair of leaflets when they transition from the delivery configuration to the deployed configuration. If the frame anchor is formed from a plastically-deformable material, the leg portions 84 can be pressed against the adjacent surfaces of the leaflets when transitioned from the delivery configuration to the deployed configuration. When configured to engage the adjacent surfaces of a pair of leaflets, the leg portions 84 can be referred to as leaflet-engagement portions of the frame anchor 80a.

In the examples of FIGS. 2-7C, each frame anchor 80a and/or the leg portions 84 thereof is positioned adjacent to (for example, immediately adjacent to and/or in contact with) the main body 53 when the frame anchor 80a is in the deployed configuration.

In other examples, at least a portion of each frame anchor 80a (for example, the leg portions 84 thereof) may be spaced apart from the main body 53 when the frame anchor 80a is in the deployed configuration. In particular, such a configuration may facilitate positioning the prosthetic heart valve 50 relative to the aortic annulus 42 such that each frame anchor 80a may engage a respective pair of native leaflets 44 with minimal obstruction by the main body 53. In some such examples, each leg portion 84 may be coupled to the main body 53 via a standoff member that spaces at least a portion of the frame anchor 80a away from the main body 53. For example, the standoff member may extend between the intermediate portion 82 and the main body 53, or the frame anchor 80a (and/or the intermediate portion 82 thereof) may include the standoff member. In some examples, each standoff member may include and/or be a spacer strut extending between the main body 53 and the intermediate portion 82.

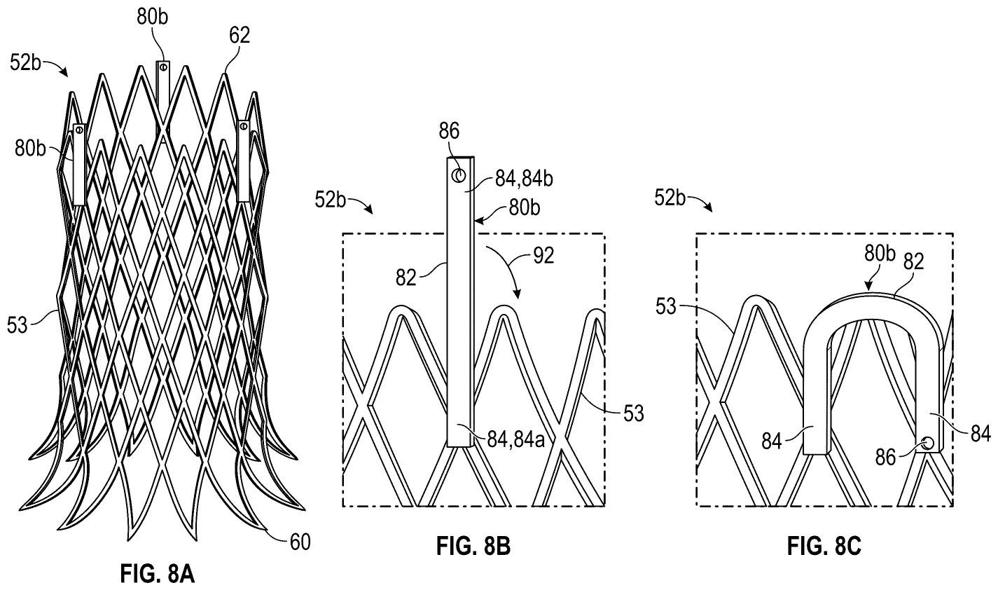

FIGS. 8A-8C illustrate a frame 52b of for a prosthetic heart valve 50, according to another example. The frame 52b comprises a main body 53 (described above) and a plurality of frame anchors 80b coupled to the main body. The frame 52b may share any suitable components, features, configurations, etc. with the frame 52a, such as the main body 53. The soft components of the prosthetic valve 50 (for example, the leaflets, an inner skirt, and/or an outer skirt) are not shown in FIGS. 8A-8C for purposes of illustration but they can be the same as described above in connection with FIG. 2. In certain examples, the frame 52b is identical to the frame 52a, except for the differences between the frame anchors 80b and the frame anchors 80a, which are described below.

Each frame anchor 80b may share any suitable components, features, configurations, etc. with the frame anchors 80a disclosed herein, except that each frame anchor 80b is connected to the main body 53 of the frame at one of the leg portions 84, rather than at the intermediate portion 82. Thus, in the example of FIGS. 8A-8C, the leg portions 84 include a first leg portion 84a connected to the main body 53 (which can be referred to as a fixed portion of the frame anchor 80b) and a second leg portion 84b that is not connected to the main body 53.

FIGS. 8A-8B illustrate each frame anchor 80b in the delivery configuration, while FIG. 8C illustrates the frame anchor 80b in the deployed configuration. In the delivery configuration, each frame anchor 80b can be straight, or substantially straight, and can extend in a downstream direction from the location where the first leg portion 84a is connected to the main body 53. In particular, and as shown in FIG. 8B, the first leg portion 84a and the second leg portion 84b can be substantially collinear when the frame anchor 80b is in the delivery configuration.

As shown in FIGS. 8B-8C, each frame anchor 80b is configured to transition from the delivery configuration to the deployed configuration via a pivoting and/or bending of the intermediate portion 82 and/or of the second leg portion 84b relative to the first leg portion 84a, in the direction indicated by arrow 92. More specifically, the intermediate portion 82 and/or the second leg portion 84b can be configured to pivot about an axis that is perpendicular to a plane that extends tangent to the main body 53 where the frame anchor 80b is attached to the main body 53. In the deployed configuration, the frame anchor 80b forms a U-shape or substantially U-shape with the ends of both leg portions 84a, 84b pointing toward the inflow end 60 of the main body 53.

As discussed above with respect to the frame anchors 80a, the frame anchors 80b can be made from a shape-memory material, wherein the frame anchors 80b can be shape set in the delivery configuration shown in FIGS. 8A and 8B. The frame anchors 80b can be held in the delivery configuration by retaining elements of a delivery apparatus, such as tethers, as further described below. Each frame anchor 80b can include an aperture 86 in leg portion 84b to receive a retaining element of the delivery apparatus. Alternatively, the anchors 80b can be made of a plastically-deformable material and can be deformed by delivery apparatus components to transition from the delivery configuration to the deployed configuration.

As discussed above, the prosthetic heart valve 50 can be delivered through a patient's vasculature to an implantation site via a delivery apparatus. FIGS. 9A-9B illustrate a distal portion of an example of a delivery apparatus 100 with the prosthetic heart valve 50 supported within the delivery apparatus 100. It is to be understood, however, that the delivery apparatus 100 also may be utilized to support and/or transport any other prosthetic heart valve according to the present disclosure. Moreover, it also is within the scope of the present disclosure that any of the prosthetic heart valves disclosed herein may be transported to the implantation site via any suitable delivery apparatus, including delivery apparatuses not specifically disclosed herein.

In the example of FIGS. 9A-9B, the delivery apparatus 100 comprises a first shaft 102 (an outer sheath or outer shaft 102 in the illustrated example), the distal end portion of which forms a delivery capsule 102d that houses the prosthetic heart valve 50 in a radially compressed state. Alternatively, the delivery capsule 102d can be separate component that is coupled to the first shaft 102. For simplicity, FIGS. 9A-9B represents the prosthetic heart valve 50 as the frame 52b; however, it is to be understood that the prosthetic heart valve 50 housed within the delivery capsule 102d also will include components such as the outer skirt 78 and the valvular structure 54 illustrated in FIG. 2.

In the example of FIGS. 9A-9B, the delivery apparatus 100 further comprises a second shaft 108 (an inner shaft 108 in the illustrated example) that extends through the prosthetic heart valve 50 and that supports a nosecone 110 of the delivery apparatus 100. Specifically, the nosecone 110 can be connected to or mounted on a distal end portion 108d of the second shaft 108. The nosecone 110 can have a tapered outer surface as shown for atraumatic tracking of the delivery apparatus 100 through a patient's vasculature. The delivery apparatus 100 further comprises a third shaft 104 (an intermediate shaft in the illustrated example), which can extend co-axially through the outer shaft 102 and over the inner shaft 108. A cord manifold 106 can be connected to the intermediate shaft 104, such as at a distal end thereof. A plurality of cords or tethers 118 can be routed through the cord manifold 106 and the frame anchors 80b to retain the frame anchors in the delivery configuration, as further described below. Proximal end portions of the outer shaft 102, the intermediate shaft 104, and the inner shaft 108 can be coupled to a handle (for example, handle 210, discussed below) of the delivery apparatus 100.

The delivery apparatus 100 is particularly suited for delivering and implanting a self-expandable prosthetic valve 50 that radially expands to its functional size under its own resiliency when deployed from the delivery capsule 102d.

Alternatively, the prosthetic heart valve 50 can be a plastically expandable prosthetic valve or a mechanically expandable heart valve. If the delivery apparatus is used to implant a plastically expandable valve, the delivery apparatus can include a balloon catheter as known in the art for expanding the prosthetic valve, such as disclosed in U.S. Publication No. 2009/0281619, which is incorporated by reference herein. If the delivery apparatus is used to implant a mechanically expandable valve, the delivery apparatus can include one or more actuators for expanding the prosthetic valve, such as disclosed in U.S. Application No. 62/945,039, filed Dec. 6, 2019, which is incorporated by reference herein.

As shown at FIGS. 9A-9B, when the delivery capsule 102d accommodates the prosthetic heart valve 50 in the radially compressed state, the delivery apparatus 100 may be connected to the prosthetic heart valve 50 via a plurality of cords or tethers 118. For purposes of illustration, the inner surface of the capsule 102d is shown to be slightly spaced from the outer surface of the frame 52b. However, it should be understood that when the prosthetic valve 50 is loaded into the capsule 102d, the inner surface of the capsule can contact the outer surface of the prosthetic valve 50 and retain the prosthetic valve in a radially compressed state, as known in the art.

In the example of FIGS. 9A-9B, each cord 118 extends distally from a proximal portion of the delivery apparatus 100 (such as handle 210) within the lumen of the outer shaft 102, through an aperture 112 in the cord manifold 106 and an aperture 86 of a corresponding frame anchor 80b and then proximally back to the proximal portion of the delivery apparatus 100. Each cord 118 may be maintained in a tensioned and/or taut state such that each frame anchor 80b is maintained in the delivery configuration while the prosthetic valve 50 is contained within the delivery capsule 102d. The ends of each cord 118 can be exposed at the proximal end of the delivery apparatus or coupled to actuators of the delivery apparatus that can control the tension of the cords and/or secure the cords relative to the handle during delivery and placement of the prosthetic valve.

The cords 118 may be made of any of various suitable biocompatible materials for use within a patient's body. In certain examples, a cord 118 can comprise a single filament cord or a multifilament or multi-strand cord formed from braiding, weaving, knitting, twisting, and wrapping a plurality of filaments or strands together. The filaments or strands can comprise polymeric fibers, such as ultra-high molecular weight polyethylene, nylon, polyester, and/or aramid, or flexible wires (for example, metal wires).

The delivery apparatus 100 may include any suitable number of cords 118. For example, the number of cords 118 may be equal to the number of frame anchors 80b of the prosthetic valve 50 (for example, three), such that each cord 118 extends through the aperture 86 of a respective frame anchor 80b. In other examples, a single cord 118 may extend through the apertures 86 of two or more frame anchors 80b of the prosthetic valve 50.

In use, the prosthetic valve 50 can be connected to the delivery apparatus 100 and loaded into the delivery capsule 102d as follows. A releasable connection can be formed between the aperture 86 of each frame anchor 80b and the cord manifold 106 with a separate cord 118. Optionally, the length of the cords 118 are selected such that the secured end of the frame is held in an at least partially radially compressed state by the cords. Additionally, the cords 118 may be configured (for example, due to their length) to exert a proximally directed restraining force on each frame anchor 80b to maintain each frame anchor 80b in the delivery configuration while the prosthetic valve 50 is loaded within the delivery capsule 102d.

After securing the end of the frame 52b with the cords 118, the delivery capsule 102d can be advanced distally over the cord manifold 106, the cords 118, and the frame 52b, causing the frame to collapse to a radially compressed state under the force of the delivery capsule 102d. The delivery capsule 102d is advanced distally until the distal end of delivery capsule 102d abuts the nosecone 110 to fully enclose the prosthetic valve 52a/52b, as shown in FIG. 9A.

After loading the prosthetic heart valve 50 within the delivery apparatus 100 as described above, the delivery apparatus can be inserted in the vasculature of a patient and advanced or navigated through the patient's vasculature to the desired implantation site (for example, through a femoral artery and the aorta when delivering the prosthetic valve 50 in a retrograde delivery approach to the native aortic valve).

Once the prosthetic valve 50 is delivered to a selected implantation site within the patient (for example, the native aortic valve), the nosecone 110 optionally can be advanced distally away from the adjacent end of the delivery capsule 102d by pushing the inner shaft 108 distally to avoid contact between the prosthetic valve and the nose cone during valve deployment. The delivery capsule 102d may be retracted in order to deploy the prosthetic valve 50. As the delivery capsule 102d is retracted, and as shown in FIG. 9B, the prosthetic valve can radially self-expand under the resiliency of the frame 52b. After the delivery capsule 102d is fully retracted from the prosthetic valve 50, the prosthetic valve is still attached to the delivery apparatus 100 by the cords 118. While still attached to the delivery apparatus, the user can manipulate the delivery apparatus (for example, by moving it in the proximal and distal directions and/or rotating it) to adjust the position of the prosthetic valve relative to the desired implantation location.

If desired, the delivery capsule can be advanced back over the prosthetic valve 50 to fully or partially recapture the prosthetic valve (bring the prosthetic valve back within the capsule) to facilitate re-positioning of the prosthetic valve or retrieval of the prosthetic valve from the patient's body. For example, after crossing the native aortic valve leaflets in a retrograde delivery approach and deploying the prosthetic valve, it may be desirable to recapture the prosthetic valve back within the capsule, retract the delivery apparatus to bring the prosthetic valve back within the aorta, and then advance the prosthetic valve back across the native aortic valve leaflets, and deploy the prosthetic valve from the capsule.

During the implantation procedure, the prosthetic valve 50 is positioned relative to the native annulus such that the inflow end portion 66 is within the native annulus and each frame anchor 80b is positioned at a location adjacent a native commissure. Imaging techniques, such as fluoroscopy, can be used to position the frame anchors relative to the native commissures and the inflow end portion relative to the native annulus. This positioning can be accomplished, for example, while the prosthetic valve is still fully contained within the capsule 102d. In other examples, the delivery capsule 102d can be partially retracted to allow at least the inflow end portion 66 to expand, while still retaining the frame anchors 80b and/or the outflow end portion 65 within the delivery capsule 102d, as shown in FIG. 9B. Final positioning of the frame anchors 80b can be performed while they are retained within the delivery capsule 102d.

Once the prosthetic valve 50 is deployed from the delivery capsule 102d and positioned at the desired implantation location, the cords 118 may be released from the frame 52b. In the example of FIGS. 9A-9B, this may be accomplished by releasing the tension in each cord 118 to release the restraining force applied to the frame anchors 80b, and then pulling one end of each cord 118 in a proximal direction, which causes the other end to be pulled distally through the outer shaft 102, the aperture 112, the aperture 86, and then back through the shaft 102 in a proximal direction, thereby releasing the cord from the frame 52b.

With the cords 118 no longer applying the restraining force to the frame anchors 80b, the frame anchors 80b are free to transition automatically from the delivery configuration to the deployed configuration, such as to extend around and/or engage the native leaflets 44 of a patient's heart 40, as depicted in FIG. 6.

The delivery apparatus 100 can be configured to deliver a prosthetic valve 50 comprising frame 52a. For use with the frame 52a, a cord 118 can be routed through the aperture 86 of each leg portion 84 of each frame anchor 80a and tensioned to place the leg portions 84 of each frame anchor 80a in the delivery configuration. The same cord 118 or separate cords 118 can be routed through the two apertures of each frame anchor 80a. The prosthetic valve 50 comprising frame 52a can be delivered and implanted in the same manner as described above, except that deploying the frame anchors 80a entails releasing two leg portions 84 of each frame anchor, which allow the leg portions of each frame anchor to revert to the deployed configuration around a pair of native leaflets.

FIGS. 10A-10B illustrate another example of a delivery apparatus 200 with the prosthetic heart valve 50 supported within the delivery apparatus 200. The delivery apparatus 200 in the illustrated example comprises a first shaft 202 (which is an outer shaft in the illustrated example), a second shaft 204 (which is an intermediate shaft in the illustrated example) extending through the first shaft, and third shaft 206 (which is an inner shaft in the illustrated example) extending through the second shaft 204. The delivery apparatus further comprises a delivery capsule 240 that houses the prosthetic heart valve 50 in a radially compressed state and a nosecone 208 supported on a distal end portion 206d of the third shaft 206. The delivery capsule 240 can be a distal end portion of the first shaft 202, or a separate component coupled to and extending from the distal end of the first shaft 202. The proximal end portions of the shafts 202, 204, and 206 can be connected to a handle 210, which can have one or more actuators, such as in the form of rotatable knobs 212, 214. The knob 212 can be configured to move the outer shaft 202 and the capsule 240 distally and proximally relative to the prosthetic valve and the other shafts 204, 206.

As shown in FIGS. 10A-10B, when the delivery capsule 240 accommodates the prosthetic heart valve 50 in the radially compressed state, the delivery apparatus 200 may be connected to the prosthetic heart valve 50 via a plurality of cords or tethers 230. The cords 230 can be formed from the same materials as described above for the cords 118. For purposes of illustration, the inner surface of the capsule 240 is shown to be slightly spaced from the outer surface of the frame 52b. However, it should be understood that when the prosthetic valve 50 is loaded into the capsule 240, the inner surface of the capsule can contact the outer surface of the prosthetic valve 50 and retain the prosthetic valve in a radially compressed state, as known in the art.

The delivery apparatus 200 further includes a cord manifold 218 coupled to the second shaft 204, as well as a plurality of release members 226. The cord manifold 218 comprises a proximal portion 220 and a distal portion 222. Each release member 226 extends between the proximal portion 220 and the distal portion 222. The proximal portion 220 is supported on a distal end portion of the second shaft 204, while the distal portion 222 is supported on a spacer shaft 224 that extends between the proximal portion 220 and the distal portion 222. The spacer shaft 224 may represent a portion of the second shaft 204 that extends between the proximal portion 220 and the distal portion 222, or the spacer shaft 224 may be a separate component from the second shaft 204. As shown in FIGS. 10A-10B, the second shaft 204 and the spacer shaft 224 may be hollow, such as to accommodate the third shaft 206 extending therethrough.

Each of the cords 230 has a first end 230a attached to the cord manifold 218, such as to the distal portion 222, and a second end 230b releasably retained by a respective release member 226. In this example, each cord 230 extends through the aperture 86 of a respective frame anchor 80b and has a second end 230b in the form of a loop that is retained on a release member 226. Each release member 226 may include and/or be any suitable structure for retaining the second end 230b of each cord 230, examples of which include a rod, a bar, a wire, a stiff cable, etc.

The release members 226 are configured to retain the cords 230 in a state connected to the frame 52b of the prosthetic valve 50 while the release members 226 extend between the proximal portion 220 and the distal portion 222 of the cord manifold 218. Each release member 226 can extend distally from the handle 210 through an aperture (not shown) in the proximal portion 220 and an aperture (not shown) in the distal portion 222. The loops 230b desirably are retained on the release members 226 at a location between the proximal portion 220 and the distal portion 222. To release the cords 230 from the prosthetic valve 50, the release members 226 may be retracted in the proximal direction so as to withdraw the release members from the distal portion 222 and optionally the proximal portion 220. Retracting the release members 226 withdraws them from the loops 230b such that they are no longer held in position by the release members 226.

In the example of FIGS. 10A-10B, three release members 226 are used, each of which retains a respective cord 230 that is coupled to a respective frame anchor 80b. In FIGS. 10A-10B, only two release members 226 and two cords 230 are visible, with the third release member and the third cord being concealed behind the inner shaft 206 and the spacer shaft 224. It should be understood, however, that any number of release members 226 and/or of cords 230 can be used.

Also, there need not be an equal number of cords 230 and release members 226. For example, the ends 230b of multiple cords 230 may be retained on a single release member. Desirably, at least three cords 230 are used to balance the attachment of the frame 52b to the cord manifold 218. In particular examples, the number of cords 230 is equal to the number of frame anchors 80b of the frame 52b of the prosthetic valve 50. Moreover, in other examples, a single cord can be used to connect the frame 52b to the cord manifold 120b at multiple locations along the outflow end of the frame by forming multiple passes extending through openings of the frame and/or the apertures 86 of the frame anchors 80b.

Each release member 226 can extend in a slideable manner through respective openings in the proximal portion 220 and the distal portion 222 of the cord manifold 218, and can be actuated to slide through the proximal portion 220 and the distal portion 222 in any suitable manner. For example, each release member 226 may have a proximal end portion that is operatively coupled to the knob 214 on the handle 210 to control movement of the release members. Each of the release members 226 is moveable in the proximal and distal directions relative to the proximal and distal portions 220, 222, of the cord manifold 218 between a distal position where each release member 226 retains a respective cord 230 and a proximal position where each release member 226 is released from a respective cord 230.

Once the prosthetic valve 50 is delivered to a selected implantation site within the patient (for example, the native aortic valve), the nosecone 208 optionally can be advanced distally away from the adjacent end of the delivery capsule 240 by advancing the inner shaft 206 distally to avoid contact between the prosthetic valve and the nose cone during valve deployment. The handle 210 can have an additional knob (not shown) to control advancement of the inner shaft 206. The delivery capsule 240 may then be retracted, such as by actuating the knob 212, in order to deploy the prosthetic valve 50. As the delivery capsule 240 is retracted, and as shown in FIG. 10B, the prosthetic valve can radially self-expand under the resiliency of the frame 52b.

After the delivery capsule 240 is fully retracted from the prosthetic valve 50, the prosthetic valve is still attached to the delivery apparatus 200 by the cords 230. Once the prosthetic valve 50 is deployed from the delivery capsule 240 and positioned at the desired implantation location, the cords 230 may be released from the frame 52b as described above. Positioning of the valve 50 and the anchors 80b can be carried out as described above in connection with FIGS. 9A-9B.

The delivery apparatus 200 can be configured to deliver a prosthetic valve 50 comprising frame 52a. For use with the frame 52a, a cord 230 can be routed through the aperture 86 of each leg portion 84 of each frame anchor 80a and releasably coupled to a release member 226 to place the leg portions 84 of each frame anchor 80a in the delivery configuration. The same cord 230 or separate cords 230 can be routed through the two apertures of each frame anchor 80a. The prosthetic valve 50 comprising frame 52a can be delivered and implanted in the same manner as described above, except that deploying the frame anchors 80a entails releasing two leg portions 84 of each frame anchor, which allow the leg portions of each frame anchor to revert to the deployed configuration around a pair of native leaflets.

Further details regarding the attachment of the prosthetic valve 10 to the delivery apparatuses 100 and 200 via one or more cords or sutures are disclosed in U.S. Publication Nos. 2014/0343670, 2012/0239142, 2010/0049313, and 2022/0000619 and in WIPO Publication No. WO2021/195090, all of which documents are incorporated by reference herein.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (e.g., with heat, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation and ultra-violet radiation. Examples of chemicals for use in sterilization include, without limitation, ethylene oxide and hydrogen peroxide. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

### Example 1

A prosthetic heart valve for implantation within a native heart valve comprising a plurality of native leaflets, the prosthetic heart valve comprising: a radially expandable frame comprising an annular main body having an inflow end and an outflow end, wherein the main body is radially expandable between a radially compressed state and a radially expanded state; and a valvular structure comprising a plurality of leaflets disposed within and coupled to the frame and configured to regulate a flow of blood in one direction through the frame from the inflow end to the outflow end; wherein the frame comprises a plurality of frame anchors coupled to the main body; and wherein each frame anchor is configured to be positioned on or around a pair of free edges of two of the native leaflets.

### Example 2

The prosthetic heart valve of any example herein, particularly example 1, wherein the prosthetic heart valve is configured to be implanted within an aortic annulus.

### Example 3

The prosthetic heart valve of any example herein, particularly any one of examples 1-2, wherein the prosthetic heart valve is a self-expanding valve that is configured to self-expand from the radially compressed state to the radially expanded state.

### Example 4

The prosthetic heart valve of any example herein, particularly any one of examples 1-3, wherein the prosthetic heart valve is configured to be expanded from the radially compressed state to the radially expanded state by inflating a balloon of a delivery apparatus.

### Example 5

The prosthetic heart valve of any example herein, particularly any one of examples 1-4, further comprising an outer skirt at least partially covering an outer surface of the main body.

### Example 6

The prosthetic heart valve of any example herein, particularly example 5, wherein the outer skirt extends at least partially between each frame anchor and the main body.

### Example 7

The prosthetic heart valve of any example herein, particularly any one of examples 1-6, wherein the main body comprises a plurality of circumferentially extending struts forming a plurality of apices at the inflow end and the outflow end of the frame.

### Example 8

The prosthetic heart valve of any example herein, particularly any one of examples 1-7, wherein, when the prosthetic heart valve is in a radially expanded state, the main body is at least substantially cylindrical proximate to the outflow end, and wherein the main body flares radially outwardly toward the inflow end.

### Example 9

The prosthetic heart valve of any example herein, particularly any one of examples 1-8, wherein the main body comprises an inflow end portion that comprises the inflow end, and wherein the inflow end portion is flared radially outward.

### Example 10

The prosthetic heart valve of any example herein, particularly any one of examples 1-9, wherein the outflow end of the main body has a first diameter, wherein the inflow end of the main body has a second diameter that is greater than the first diameter, and wherein the second diameter is greater than a diameter of an annulus within which the prosthetic heart valve is implanted.

### Example 11

The prosthetic heart valve of any example herein, particularly any one of examples 1-10, wherein each frame anchor is configured to transition from a delivery configuration to a deployed configuration to anchor the prosthetic heart valve within the native heart valve.

### Example 12

The prosthetic heart valve of any example herein, particularly example 11, wherein each frame anchor is configured to bend as the frame anchor transitions between the delivery configuration and the deployed configuration.

### Example 13

The prosthetic heart valve of any example herein, particularly any one of examples 11-12, wherein each frame anchor is biased toward the deployed configuration.

### Example 14

The prosthetic heart valve of any example herein, particularly any one of examples 11-13, wherein each frame anchor is configured to automatically transition from the delivery configuration to the deployed configuration when a restraining force is removed from the frame anchor.

### Example 15

The prosthetic heart valve of any example herein, particularly any one of examples 11-14, wherein each frame anchor comprises at least one aperture that is configured to engage a cord of a delivery apparatus, wherein each frame anchor is configured to receive a restraining force from the cord, wherein each frame anchor is configured to be maintained in the delivery configuration while the cord applies the restraining force to the frame anchor, and wherein each frame anchor is free to transition from the delivery configuration to the deployed configuration when the cord no longer applies the restraining force to the frame anchor.

### Example 16

The prosthetic heart valve of any example herein, particularly any one of examples 11-15, wherein each frame anchor is substantially U-shaped when the frame anchor is in the deployed configuration.

### Example 17

The prosthetic heart valve of any example herein, particularly any one of examples 1-16, wherein the frame anchors are configured to engage the native leaflets to anchor the prosthetic heart valve in position relative to an annulus of a heart comprising the native heart valve against retrograde blood flow.

### Example 18

The prosthetic heart valve of any example herein, particularly any one of examples 1-17, wherein the main body comprises a plurality of circumferentially extending struts forming a plurality of apices at the inflow end and the outflow end of the frame, and wherein each frame anchor is coupled to the main body at an intersection of struts between a pair of circumferentially adjacent apices.

### Example 19

The prosthetic heart valve of any example herein, particularly any one of examples 1-18, wherein each frame anchor is formed of a plastically deformable material.

### Example 20

The prosthetic heart valve of any example herein, particularly any one of examples 1-19, wherein each frame anchor is formed of a shape-memory material.

### Example 21

The prosthetic heart valve of any example herein, particularly any one of examples 1-20, wherein each frame anchor is formed of the same material as at least a portion of the main body.

### Example 22

The prosthetic heart valve of any example herein, particularly any one of examples 1-21, wherein each frame anchor is integrally formed with at least a portion of the main body.

### Example 23

The prosthetic heart valve of any example herein, particularly any one of examples 1-21, wherein each frame anchor is formed separately from the main body and joined to the main body.

### Example 24

The prosthetic heart valve of any example herein, particularly any one of examples 1-23, wherein each frame anchor is coupled to the main body via one or more of a weld, an adhesive, a suture, and a mechanical connector.

### Example 25

The prosthetic heart valve of any example herein, particularly any one of examples 1-24, wherein the prosthetic heart valve is configured such that, when the prosthetic heart valve is implanted within the native heart valve, each frame anchor engages a respective pair of native leaflets of the native heart valve proximate to a native commissure of the native heart valve.

### Example 26

The prosthetic heart valve of any example herein, particularly any one of examples 1-25, wherein the prosthetic heart valve is configured such that, when the prosthetic heart valve is implanted within the native heart valve, each frame anchor clamps onto a respective pair of native leaflets of the native heart valve.

### Example 27

The prosthetic heart valve of any example herein, particularly any one of examples 1-26, wherein each frame anchor comprises an intermediate portion and a pair of leg portions extending away from the intermediate portion.

### Example 28

The prosthetic heart valve of any example herein, particularly any example 27, wherein the prosthetic heart valve is configured such that, when the prosthetic heart valve is implanted within the native heart valve, each leg portion engages a native leaflet of the native heart valve proximate to a native commissure of the native heart valve.

### Example 29

The prosthetic heart valve of any example herein, particularly any one of examples 27-28, wherein each leg portion is integrally formed with the intermediate portion.

### Example 30

The prosthetic heart valve of any example herein, particularly any one of examples 27-29, wherein, for each frame anchor, the intermediate portion is fixed in position relative to the main body, and wherein each leg portion is configured to pivot relative to the intermediate portion to transition the frame anchor between a delivery configuration and a deployed configuration.

### Example 31

The prosthetic heart valve of any example herein, particularly example 30, wherein each leg portion is configured to pivot about an axis that is perpendicular to a plane that extends tangent to the main body where the frame anchor is attached to the main body.

### Example 32

The prosthetic heart valve of any example herein, particularly any one of examples 30-31, wherein, when the frame anchor is in the delivery configuration, each leg portion extends away from the intermediate portion toward the outflow end, and wherein, when the frame anchor is in the deployed configuration, each leg portion extends away from the intermediate portion toward the inflow end.

### Example 33

The prosthetic heart valve of any example herein, particularly example 32, wherein, when the frame anchor is in the delivery configuration, each leg portion extends to the outflow end and continues beyond the outflow end.

### Example 34

The prosthetic heart valve of any example herein, particularly any one of examples 30-33, wherein each leg portion comprises an aperture that is configured to engage a cord of a delivery apparatus.

### Example 35

The prosthetic heart valve of any example herein, particularly any one of examples 30-34, wherein the frame anchor is substantially U-shaped when the frame anchor is in the delivery configuration.

### Example 36

The prosthetic heart valve of any example herein, particularly any one of examples 27-29, wherein, for each frame anchor, the pair of leg portions comprises a first leg portion and a second leg portion, wherein the first leg portion is fixed in position relative to the main body, and wherein one or both of the intermediate portion and the second leg portion are configured to pivot relative to the first leg portion to transition the frame anchor between a delivery configuration and a deployed configuration.

### Example 37

The prosthetic heart valve of any example herein, particularly example 36, wherein the intermediate portion is configured to pivot about an axis that is perpendicular to a plane that extends tangent to the main body where the frame anchor is attached to the main body.

### Example 38

The prosthetic heart valve of any example herein, particularly any one of examples 36-37, wherein, when the frame anchor is in the delivery configuration, the second leg portion extends away from the first leg portion toward the outflow end; and wherein, when the frame anchor is in the deployed configuration, the second leg portion extends away from the first leg portion toward the inflow end.

### Example 39

The prosthetic heart valve of any example herein, particularly example 38, wherein, when the frame anchor is in the delivery configuration, the second leg portion extends to the outflow end and continues beyond the outflow end.

### Example 40

The prosthetic heart valve of any example herein, particularly any one of examples 36-39, wherein the second leg portion is not connected to the main body.

### Example 41

The prosthetic heart valve of any example herein, particularly any one of examples 36-40, wherein the second leg portion comprises an aperture that is configured to engage a cord of a delivery apparatus.

### Example 42

The prosthetic heart valve of any example herein, particularly any one of examples 36-41, wherein the first leg portion and the second leg portion are substantially collinear when the frame anchor is in the delivery configuration.

### Example 43

A prosthetic heart valve for implantation within a native heart valve comprising a plurality of native leaflets, the prosthetic heart valve comprising: a radially expandable frame comprising an annular main body having an inflow end and an outflow end, wherein the main body is radially expandable between a radially compressed state and a radially expanded state; and a valvular structure comprising a plurality of leaflets disposed within and coupled to the frame and configured to regulate a flow of blood in one direction through the frame from the inflow end to the outflow end; wherein the frame comprises a plurality of frame anchors coupled to the main body, wherein each frame anchor comprises a first leg portion, a second leg portion, and an intermediate portion, wherein each of the first leg portion and the second leg portion extends away from the intermediate portion; wherein each frame anchor is configured to bend relative to the main body to transition between a delivery configuration and a deployed configuration, and wherein, for each frame anchor, when the frame anchor is in the deployed configuration and when the prosthetic heart valve is implanted within the native heart valve, the first leg portion extends alongside a first leaflet of the plurality of native leaflets, the second leg portion extends alongside a second leaflet of the plurality of native leaflets, and the intermediate portion is positioned adjacent to free edges of each of the first leaflet and the second leaflet.

### Example 44

The prosthetic heart valve of any example herein, particularly example 43, wherein each frame anchor is substantially U-shaped when the frame anchor is in the deployed configuration.

### Example 45

The prosthetic heart valve of any example herein, particularly any one of examples 43-44, wherein, for each frame anchor, the first leg portion and the second leg portion extend at least substantially parallel to one another when the frame anchor is in the delivery configuration and when the frame anchor is in the deployed configuration.

### Example 46

The prosthetic heart valve of any example herein, particularly any one of examples 43-45, wherein, for each frame anchor, when the frame anchor is in the deployed configuration and when the prosthetic heart valve is implanted within the native heart valve, the first leg portion presses against the first leaflet, and the second leg portion presses against the second leaflet.

### Example 47

The prosthetic heart valve of any example herein, particularly any one of examples 43-46, wherein each frame anchor is substantially U-shaped when the frame anchor is in the delivery configuration.

### Example 48

The prosthetic heart valve of any example herein, particularly any one of examples 43-47, wherein, for each frame anchor, the first leg portion and the second leg portion are substantially collinear when the frame anchor is in the delivery configuration.

### Example 49

The prosthetic heart valve of any example herein, particularly any one of examples 43-48, wherein, for each frame anchor, one or both of the first leg portion and the second leg portion comprises an aperture that is configured to engage a cord of a delivery apparatus.

### Example 50

The prosthetic heart valve of any example herein, particularly any one of examples 43-49, wherein, for each frame anchor, the intermediate portion is fixedly coupled to the main body.

### Example 51

The prosthetic heart valve of any example herein, particularly any one of examples 43-50, wherein, for each frame anchor, the first leg portion is fixedly coupled to the main body.

### Example 52

The prosthetic heart valve of any example herein, particularly any one of examples 1-51, wherein each frame anchor is at least partially covered by a protective covering.

### Example 53

A prosthetic heart valve delivery assembly, comprising: a prosthetic heart valve for implantation within a native heart valve comprising a plurality of native leaflets, the prosthetic heart valve comprising: a radially expandable frame comprising an annular main body having an inflow end and an outflow end, wherein the main body is radially expandable between a radially compressed state and a radially expanded state; and a valvular structure comprising a plurality of leaflets disposed within and coupled to the frame and configured to regulate a flow of blood in one direction through the frame from the inflow end to the outflow end; wherein the frame comprises a plurality of frame anchors coupled to the main body, and wherein each frame anchor is configured to be positioned on or around a pair of free edges of two of the native leaflets; and a delivery apparatus for delivering the prosthetic heart valve to an implantation site, the delivery apparatus comprising: a delivery capsule configured to house the prosthetic heart valve in a radially compressed state; and a plurality of cords, each configured to be releasably connected to a respective frame anchor of the prosthetic heart valve and to apply a restraining force to the respective frame anchor.

### Example 54

The prosthetic heart valve delivery assembly of any example herein, particularly example 53, wherein, for each cord and the respective frame anchor, the frame anchor comprises an aperture, and the cord extends through the aperture when the prosthetic heart valve is housed within the delivery capsule.

### Example 55

The prosthetic heart valve delivery assembly of any example herein, particularly any one of examples 53-54, wherein each frame anchor is configured to transition from a delivery configuration to a deployed configuration to anchor the prosthetic heart valve within the native heart valve, and wherein, for each cord and the respective frame anchor, the cord is configured to maintain the frame anchor in the delivery configuration while the cord applies the restraining force to the frame anchor, and the frame anchor automatically transitions from the delivery configuration to the deployed configuration when the cord no longer applies the restraining force to the frame anchor.

### Example 56

The prosthetic heart valve delivery assembly of any example herein, particularly any one of examples 53-55, wherein the cords maintain an end of the frame that comprises the frame anchors in an at least partially radially compressed state when the cords are connected to the frame anchors and apply the restraining force to the frame anchors.

### Example 57

The prosthetic heart valve delivery assembly of any example herein, particularly any one of examples 53-56, wherein the delivery apparatus further comprises: a handle portion; a first shaft extending between the handle portion and the delivery capsule; an inner shaft configured to extend through the prosthetic heart valve when the prosthetic heart valve is housed within the delivery capsule; and a nosecone mounted on a distal end portion of the inner shaft distal to the delivery capsule.

### Example 58

The prosthetic heart valve delivery assembly of any example herein, particularly example 57, further comprising: a cord manifold comprising a proximal portion and a distal portion axially spaced apart from one another; and a plurality of release members extending from the handle portion through the first shaft, through the proximal portion, and at least partially through the distal portion; wherein each cord comprises a first end attached to the proximal portion and a second end retained on a respective release member between the proximal portion and the distal portion; and wherein the delivery apparatus is configured such that withdrawing the release members from one or both of the distal portion and the proximal portion such that the second ends of the cords are no longer retained in position by the release members.

### Example 59

The prosthetic heart valve delivery assembly of any example herein, particularly example 58, wherein each release member comprises a proximal end portion that is coupled to a knob on the handle portion to control movement of the release members.

### Example 60

The prosthetic heart valve delivery assembly of any example herein, particularly any one of examples 58-59, wherein each release member is moveable in the proximal and distal directions relative to the proximal portion and the distal portion of the cord manifold between a distal position, in which each release member retains a respective cord, and a proximal position, in which each release member is released from the respective cord.

### Example 61

The prosthetic heart valve delivery assembly of any example herein, particularly any one of examples 58-60, wherein each second end comprises a loop that extends around a respective release member.

### Example 62

The prosthetic heart valve delivery assembly of any example herein, particularly any one of examples 58-61, wherein each release member comprises one or more of a rod, a bar, a wire, and a cable.

### Example 63

The prosthetic heart valve delivery assembly of any example herein, particularly any one of examples 57-62, wherein the delivery apparatus is configured such that the prosthetic heart valve may be released from the delivery capsule by one or both of: advancing the nosecone distally away from an adjacent end of the delivery capsule by advancing the inner shaft distally of the handle portion; and retracting the delivery capsule proximally toward the handle portion.

### Example 64

The prosthetic heart valve of any example herein, particularly any one of examples 1-52, wherein the prosthetic heart valve is sterilized.

### Example 65

The prosthetic heart valve delivery assembly of any example herein, particularly any one of examples 53-63, wherein the delivery apparatus and the prosthetic heart valve are sterilized.

### Example 66

A method comprising sterilizing the prosthetic heart valve or the prosthetic heart valve delivery assembly of any example herein, particularly any one of examples 1-65.

The features described herein with regard to any example can be combined with other features described in any one or more of the other examples, unless otherwise stated. For example, any one or more of the features of one prosthetic heart valve can be combined with any one or more features of another prosthetic heart valve. As another example, any one or more features of one prosthetic heart valve delivery assembly can be combined with any one or more features of another prosthetic heart valve delivery assembly.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. A prosthetic heart valve for implantation within a native heart valve comprising a plurality of native leaflets, the prosthetic heart valve comprising:
a radially expandable frame comprising an annular main body having an inflow end and an outflow end, wherein the main body is radially expandable between a radially compressed state and a radially expanded state; and
a valvular structure comprising a plurality of leaflets disposed within and coupled to the frame and configured to regulate a flow of blood in one direction through the frame from the inflow end to the outflow end;
wherein the frame comprises a plurality of frame anchors coupled to the main body; and
wherein each frame anchor is configured to be positioned on or around a pair of free edges of two of the native leaflets.

2. The prosthetic heart valve of claim 1, wherein the frame anchors are configured to engage the native leaflets to anchor the prosthetic heart valve in position relative to an annulus of the native heart valve against retrograde blood flow.

3. The prosthetic heart valve of claim 1, further comprising an outer skirt at least partially covering an outer surface of the main body; wherein the outer skirt extends at least partially between each frame anchor and the main body.

4. The prosthetic heart valve of claim 1, wherein the main body comprises an inflow end portion that comprises the inflow end, and wherein the inflow end portion is flared radially outward.

5. The prosthetic heart valve of claim 1, wherein each frame anchor is configured to transition from a delivery configuration to a deployed configuration to anchor the prosthetic heart valve within the native heart valve, and wherein each frame anchor is biased toward the deployed configuration.

6. The prosthetic heart valve of claim 5, wherein each frame anchor is configured to bend as the frame anchor transitions between the delivery configuration and the deployed configuration.

7. The prosthetic heart valve of claim 5, wherein each frame anchor comprises at least one aperture that is configured to engage a cord of a delivery apparatus, wherein each frame anchor is configured to receive a restraining force from the cord, wherein each frame anchor is configured to be maintained in the delivery configuration while the cord applies the restraining force to the frame anchor, and wherein each frame anchor is free to transition from the delivery configuration to the deployed configuration when the cord no longer applies the restraining force to the frame anchor.

8. The prosthetic heart valve of claim 5, wherein each frame anchor is substantially U-shaped when the frame anchor is in one or both of the deployed configuration and the delivery configuration.

9. The prosthetic heart valve of claim 1, wherein the prosthetic heart valve is configured such that, when the prosthetic heart valve is implanted within the native heart valve, each frame anchor engages a respective pair of native leaflets of the native heart valve proximate to a native commissure of the native heart valve.

10. The prosthetic heart valve of claim 1, wherein each frame anchor comprises an intermediate portion and a pair of leg portions extending away from the intermediate portion, and wherein the prosthetic heart valve is configured such that, when the prosthetic heart valve is implanted within the native heart valve, each leg portion engages a native leaflet of the native heart valve proximate to a native commissure of the native heart valve.

11. The prosthetic heart valve of claim 10, wherein, for each frame anchor, the intermediate portion is fixed in position relative to the main body, and wherein each leg portion is configured to pivot relative to the intermediate portion to transition the frame anchor between a delivery configuration and a deployed configuration.

12. The prosthetic heart valve of claim 11, wherein, when the frame anchor is in the delivery configuration, each leg portion extends away from the intermediate portion toward the outflow end, and wherein, when the frame anchor is in the deployed configuration, each leg portion extends away from the intermediate portion toward the inflow end.

13. The prosthetic heart valve of claim 10, wherein, for each frame anchor, the pair of leg portions comprises a first leg portion and a second leg portion, wherein the first leg portion is fixed in position relative to the main body, and wherein one or both of the intermediate portion and the second leg portion are configured to pivot relative to the first leg portion to transition the frame anchor between a delivery configuration and a deployed configuration.

14. The prosthetic heart valve of claim 13, wherein the second leg portion comprises an aperture that is configured to engage a cord of a delivery apparatus.

15. A prosthetic heart valve for implantation within a native heart valve comprising a plurality of native leaflets, the prosthetic heart valve comprising:
a radially expandable frame comprising an annular main body having an inflow end and an outflow end, wherein the main body is radially expandable between a radially compressed state and a radially expanded state; and
a valvular structure comprising a plurality of leaflets disposed within and coupled to the frame and configured to regulate a flow of blood in one direction through the frame from the inflow end to the outflow end;
wherein the frame comprises a plurality of frame anchors coupled to the main body, wherein each frame anchor comprises a first leg portion, a second leg portion, and an intermediate portion, wherein each of the first leg portion and the second leg portion extends away from the intermediate portion;
wherein each frame anchor is configured to bend relative to the main body to transition between a delivery configuration and a deployed configuration, and wherein, for each frame anchor, when the frame anchor is in the deployed configuration and when the prosthetic heart valve is implanted within the native heart valve, the first leg portion extends alongside a first leaflet of the plurality of native leaflets, the second leg portion extends alongside a second leaflet of the plurality of native leaflets, and the intermediate portion is positioned adjacent to free edges of each of the first leaflet and the second leaflet.

16. The prosthetic heart valve of claim 15, wherein each frame anchor is substantially U-shaped when the frame anchor is in one or both of the deployed configuration and the delivery configuration.

17. The prosthetic heart valve of claim 15, wherein, for each frame anchor, when the frame anchor is in the deployed configuration and when the prosthetic heart valve is implanted within the native heart valve, the first leg portion presses against the first leaflet, and the second leg portion presses against the second leaflet.

18. The prosthetic heart valve of claim 15, wherein, for each frame anchor, one or both of the first leg portion and the second leg portion comprises an aperture that is configured to engage a cord of a delivery apparatus.

19. A prosthetic heart valve delivery assembly, comprising:
a prosthetic heart valve for implantation within a native heart valve comprising a plurality of native leaflets, the prosthetic heart valve comprising:
a radially expandable frame comprising an annular main body having an inflow end and an outflow end, wherein the main body is radially expandable between a radially compressed state and a radially expanded state; and
a valvular structure comprising a plurality of leaflets disposed within and coupled to the frame and configured to regulate a flow of blood in one direction through the frame from the inflow end to the outflow end;
wherein the frame comprises a plurality of frame anchors coupled to the main body, and wherein each frame anchor is configured to be positioned on or around a pair of free edges of two of the native leaflets; and
a delivery apparatus for delivering the prosthetic heart valve to an implantation site, the delivery apparatus comprising:
a delivery capsule configured to house the prosthetic heart valve in a radially compressed state; and
a plurality of cords, each configured to be releasably connected to a respective frame anchor of the prosthetic heart valve and to apply a restraining force to the respective frame anchor.

20. The prosthetic heart valve delivery assembly of claim 19, wherein, for each cord and the respective frame anchor, the frame anchor comprises an aperture, and the cord extends through the aperture when the prosthetic heart valve is housed within the delivery capsule.
